(19) United States
(12) Patent Application Publication
(10) Pub. No.: US 2021/0161425 A1
(43) Pub. Date: Jun. 3, 2021

(54) POSITIONING SYSTEM FOR SWALLOWABLE DEVICE AND METHOD THEREOF

(71) Applicants: ANKON TECHNOLOGIES CO., LTD, Wuhan (CN); ANX IP HOLDING PTE. LTD., SINGAPORE (SG)

(72) Inventors: Tianyi YANGDAI, Wuhan (CN); Wei WU, WUHAN (CN); Hao LIU, WUHAN (CN); HANGYU PENG, Wuhan (CN); YANLI LIU, Wuhan (CN); FANHUA MING, WUHAN (CN)

(73) Assignees: ANKON TECHNOLOGIES CO., LTD, Wuhan (CN); ANX IP HOLDING PTE. LTD., SINGAPORE (SG)

(21) Appl. No.: 17/106,019

(22) Filed: Nov. 27, 2020

(30) Foreign Application Priority Data
Nov. 28, 2019 (CN) 201911188050.6

(51) Int. Cl. A61B 5/06

(52) U.S. Cl. CPC A61B 5/062; A61B 2562/0219; A61B 2562/0223

(57) ABSTRACT

A positioning method for a swallowable device is provided for positioning the in vivo swallowable device. The method includes: entering the m-th positioning period, m≤1; energizing p electromagnetic coils simultaneously or sequentially, p≤2; obtaining the actual magnetic field information Bwt sensed by the swallowable device each time the electromagnetic coils under energized, 1≥t≥p; calculating the individual magnetic field information Bi at the swallowable device when the electromagnetic coils are separately energized, 1≥i≥p; detecting the pitch angle and roll angle of the swallowable device in the m-th positioning period; calculating the yaw angle and spatial coordinates of the swallowable device in the m-th positioning period according to the pitch angle and roll angle and the individual magnetic field information Bi.

### CROSS-REFERENCE OF RELATED APPLICATIONS

The application claims priority to Chinese Patent Application No. 201911188050.6 filed on Nov. 28, 2019, the contents of which are incorporated by reference herein.

### FIELD OF INVENTION

The present invention relates to a technique for a swallowable device, and more particularly to a positioning system for a swallowable device and a method thereof.

### BACKGROUND

Real-time determination of the three-dimensional position and posture of a swallowable device, for example a capsule-shaped device, inside the human body is of great significance in capsule control and disease positioning. At present, Numerous capsule positioning methods have been proposed at home and abroad, which can be roughly divided into four aspects: RF-based positioning systems, magnetic field-based positioning systems, vision-based positioning systems, and other positioning systems.

Among them, the magnetic field-based positioning system is not subject to human body interference and is also harmless to human body. In this technical system, it can be divided into a transmitting-type magnetic field-based positioning method and a receiving-type magnetic field-based positioning method.

In the transmitting-type magnetic field-based positioning method, a small permanent magnet or electromagnetic coil is disposed inside the capsule, and a magnetic sensor array is arranged outside the body. The magnetic signal of the capsule is detected by the magnetic sensor array, and the position and posture of the capsule can be reconstructed by magnetic field modeling and nonlinear solution. In this method, due to the confined space and limited energy supply inside the capsule, the static magnetic or electromagnetic fields that can be generated are very weak, so the detection distance, that is, the distance between the capsule and the magnetic sensor array is usually difficult to exceed 15 cm. In addition, for a system that controls the in vivo movement of a capsule by a magnetic controlled device, the magnetic field strength of the device is much greater than that of the capsule, and changes over time, which may reduce the accuracy and practicability of the transmitting-type positioning.

In the receiving-type magnetic field-based positioning method, a magnetic field sensor is disposed in the capsule to receive magnetic field signals transmitted from an external magnetic field source which may be a permanent magnet, an electromagnetic coil, or a combination of the two, and the position and posture of the capsule can be obtained through calculation. Since the signal source is external, the signal strength can be enhanced by various means so that the capsule can detect a magnetic field signal with a sufficient signal-to-noise ratio for positioning, which increases the detection range and measurement accuracy of the system. However, similarly, for a system that controls the in vivo movement of a capsule by a magnetic control device, the magnetic field of the magnetic control device can also interfere with the external magnetic field source, so it is impossible to accurately obtain the capsule position and posture.

Therefore, it is necessary to design a positioning system for a swallowable device and a method thereof, which can not be interfered by the external magnetic field.

### SUMMARY OF THE INVENTION

To solve one of the above-mentioned problems, the present invention discloses a positioning method for a swallowable device, which is used for the in vivo positioning of the swallowable device. The method comprises: entering the m-th positioning period, wherein m≥1; energizing p electromagnetic coils simultaneously or sequentially, wherein, p>2; obtaining the actual magnetic field information Bwt sensed by the swallowable device each time the electromagnetic coils under energized state, wherein,1≤t≤p; calculating the individual magnetic field information Bi at the swallowable device within the electromagnetic coils under separately energized state, wherein, 1≤i≤p; detecting the pitch angle and roll angle of the swallowable device in the m-th positioning period; calculating the yaw angle and spatial coordinates of the swallowable device in the m-th positioning period according to the pitch angle and roll angle and the individual magnetic field information Bi.

In an embodiment, before the step “calculating the individual magnetic field information Bi at the swallowable device within the electromagnetic coils under separately energized state” comprises: detecting the bottom magnetic field information Bg sensed by the swallowable device when the electromagnetic coils are not energized in the m-th positioning period; and the step “calculating the individual magnetic field information Bi at the swallowable device within the electromagnetic coils under separately energized state” comprises: calculating the individual magnetic field information Bi at the swallowable device within the electromagnetic coils under separately energized state according to the bottom magnetic field information Bg and the actual magnetic field information Bwt.

In an embodiment, the step “energizing p electromagnetic coils simultaneously or sequentially” comprises: conducting positive or negative pulse signals sequentially in the p electromagnetic coils, at most one of the electromagnetic coils under energized state at the same time; and the step “calculating the individual magnetic field information Bi at the swallowable device within the electromagnetic coils under separately energized state according to the bottom magnetic field information Bg and the actual magnetic field information Bwt” comprises: Bi=Bwt−Bg, wherein, t=i.

In an embodiment, the step “energizing p electromagnetic coils simultaneously or sequentially” comprises: conducting periodic signals in the p electromagnetic coils simultaneously, and the current frequency conducted in the electromagnetic coils is different; and the step “calculating the individual magnetic field information Bi at the swallowable device within the electromagnetic coils under separately energized state” comprises: calculating the magnetic field information Bwt by Fourier decomposition to obtain the individual magnetic field information Bi at the swallowable device within the p electromagnetic coils under separately energized state.

In an embodiment, the step “energizing p electromagnetic coils simultaneously or sequentially” comprises: controlling to conduct square-wave pulse signals in the p electromagnetic coils sequentially, the absolute values of high level and low level in the same square-wave pulse signal are equal, and at most one of the electromagnetic coils under energized state at the same time; and the step “obtaining the actual magnetic field information Bwt sensed by the swallowable device each time the electromagnetic coils under energized state” comprises: obtaining the actual magnetic field information Bwt₊ sensed by the swallowable device each time the electromagnetic coils conducts a positive current; obtaining the actual magnetic field information Bwt₋ sensed by the swallowable device each time the electromagnetic coils conducts a negative current; and the step “calculating the individual magnetic field information Bi at the swallowable device within the electromagnetic coils under separately energized state” comprises:

$$B i = \frac{B w {t}_{+} - {Bwt}_{-}}{2} ,$$

wherein, t=i.

In an embodiment, the step “obtaining the actual magnetic field information Bwt sensed by the swallowable device each time the electromagnetic coils under energized state” comprises: appling square-wave pulse signals with the same frequency to the p electromagnetic coils simultaneously, the absolute values of the high level and low level in the same square-wave pulse signal are equal, and the phases of square waves applied to the electromagnetic coils are different; and the step “calculating the individual magnetic field information Bi at the swallowable device within the electromagnetic coils under separately energized state” comprises: decomposing the actual magnetic field information Bwt according to the difference in amplitude, and obtaining Bwtₙ in turn; wherein 1≤n≤2p;

$$B i = \frac{{Bwt}_{j} - Bw {t}_{k}}{2} ,$$

wherein, j and k belong to n.

In an embodiment, the step “calculating the yaw angle and spatial coordinates of the swallowable device in the m-th positioning period according to the pitch angle and roll angle and the individual magnetic field information Bi” comprises: establishing a magnetic field model for the electromagnetic coil, and the magnetic field model is:

$${B}_{t} = \int \frac{{μ}_{0} I}{4 π} \frac{dI \times r}{{r}^{3}} , \qquad ( Formula 1 )$$

wherein, μ₀ is the vacuum permeability, I is the current intensity in the coil and corresponds to the measured individual magnetic field information Bi, dl is the current element, r is the vector from the current element to the swallowable device, r is the distance from the current element to the swallowable device; calculating the estimated magnetic field information Bti at the location of the swallowable device within the electromagnetic coils under energized state according to Formula 1; establishing a spatial coordinates calculation model, that is:

$${argmin}_{x , y , z , Yaw} {\sum }_{i = 1}^{p} {‖ {R}_{r} {R}_{p} R ( Yaw ) {B}_{i} - {B}_{ti} ( x , y , z ) ‖}_{2}^{2} , \qquad ( Formula 2 )$$

wherein, Rᵣ is the rotation matrix corresponding to the roll angle, Rₚ is the rotation matrix corresponding to the pitch angle, R_{Yaw} is the rotation matrix corresponding to the yaw angle; performing an optimized solution algorithm on the calculation model in Formula 2, and calculating the yaw angle and spatial coordinates of the swallowable device in the m-th positioning period.

In an embodiment, before the step “detecting the pitch angle and roll angle of the swallowable device in the m-th positioning period” comprises: determining whether the swallowable device moves in the m-th positioning period; entering the detection or calculation of the pitch angle and roll angle when the swallowable device does not move in the m-th positioning period; entering the m+1-th positioning period when the swallowable device moves in the m-th positioning period.

In an embodiment, the step “determining whether the swallowable device moves in the m-th positioning period” comprises: counting the vibration amplitude of the acceleration value of the swallowable device in the m-th positioning period; determining that the capsule moves when the vibration amplitude is greater than the threshold S; determining that the capsule does not move when the vibration amplitude is less than or equal to the threshold S.

In an embodiment, the positioning method further comprises: obtaining the spatial coordinates of the swallowable device in the m-th, m+1-th, and m+2-th positioning periods, and recording them as A, B, and C respectively; predicting the spatial coordinates D′ of the swallowable device in the m+3-th positioning period; entering the m+3-th positioning period, and calculating the spatial coordinates D of the swallowable device in the m+3-th positioning period; calculating the distance d between D and D′ in three-dimensional space; if d<threshold L, using D as the spatial coordinates of the swallowable device in the m+3-th positioning period; if d>threshold L, using D′ as the spatial coordinates of the swallowable device in the m+3-th positioning period.

In an embodiment, the step “predicting the spatial coordinate D′ of the swallowable device in the m+3-th positioning period” comprises: D′=C+2BC−AB, wherein, BC and AB are both vectors.

In an embodiment of the present invention, the positioning method further comprises:

determining the magnitude of the individual magnetic field information Bi in the m-th period; if gₕ<|Bi|, reducing the current of the electromagnetic coil i in the m+1-th period; if gₗ<|Bi|gₕ, keeping the current of the electromagnetic coil i in the m+1-th period; if |Bi|<gₗ, increasing the current of the electromagnetic coil i in the m+1-th period.

In an embodiment, the step “energizing p electromagnetic coils simultaneously or sequentially” comprises: selecting p electromagnetic coils from n electromagnetic coils, and energizing simultaneously or sequentially, wherein, n>p; the positioning method further comprises: changing the position or quantity of the energized electromagnetic coils in different positioning periods according to the position of the swallowable device.

The present invention further provides a positioning system for a swallowable device using the method described above, used for positioning the in vivo swallowable device, such as a capsule endoscope, comprising a swallowable device and a positioning device for detecting the position of the swallowable device. The swallowable device comprises an enclosure, an attitude angle sensor disposed in the enclosure for detecting the attitude angle of the swallowable device, and a magnetic field sensor disposed in the enclosure for collecting magnetic field information. The positioning device comprises an examination surface and at least two electromagnetic coils. The axes of the electromagnetic coils are parallel to each other and arranged perpendicular to the examination surface.

In an embodiment, the swallowable device further comprises a magnet, and the positioning device further comprises a magnetic control device for controlling the magnet.

In an embodiment, the electromagnetic coils are located on the side of the examination surface away from the swallowable device, and the magnetic control device and the swallowable device are on the same side of the examination surface.

In an embodiment, the electromagnetic coils are arranged in a straight line.

In an embodiment, the electromagnetic coils are arranged in a matrix.

In an embodiment, the positioning system for the swallowable device comprises a cooling device that is arranged between the examination surface and the electromagnetic coils.

Compared with the prior art, the positioning system and method for a swallowable device disclosed herein can first calculate the actual magnetic field information Bwt, and calculate the individual magnetic field information Bi in the subsequent process, thereby eliminating the influence of the bottom magnetic field information Bg on the swallowable device. Moreover, in the present invention, the pitch angle (pitch) and the roll angle (roll) are first measured, and then the yaw angle (yaw) and the spatial coordinates are calculated according to the above information. The calculation method is more concise, with lower noise and better effect. In addition, there is no need to initialize the posture of the swallowable device, and the positioning algorithm does not rely on vertical result, but is only turned on for use when needed to obtain the positioning result.

### BRIEF DESCRIPTION OF THE DRAWINGS

FIG. 1 is a schematic diagram of a positioning system for a swallowable device of the present invention.

FIG. 2 is a schematic diagram of the individual magnetic field information Bi and the actual magnetic field information Bwi according to the first embodiment of the present invention.

FIG. 3 is a schematic diagram of the individual magnetic field information Bi and the actual magnetic field information Bwi according to the second embodiment of the present invention.

FIG. 4 is a schematic diagram of the individual magnetic field information Bi and the actual magnetic field information Bwi according to the third embodiment of the present invention.

FIG. 5 is a schematic diagram of the individual magnetic field information Bi and the actual magnetic field information Bwi according to the fourth embodiment of the present invention.

FIG. 6 is a schematic diagram of the structure of the electromagnetic coils arranged in a matrix in the present invention.

### DETAILED DESCRIPTION

In order to enable those skilled in the art to better understand the technical solutions disclosed, the present invention can be clearly and completely described in detail below with reference to the accompanying drawings and preferred embodiments. Obviously, the described embodiments are only a part of the embodiments of the present invention, rather than all the embodiments. All other embodiments obtained by those having ordinary skill in the art without creative work based on the embodiments of the present invention are included in the scope of the present invention.

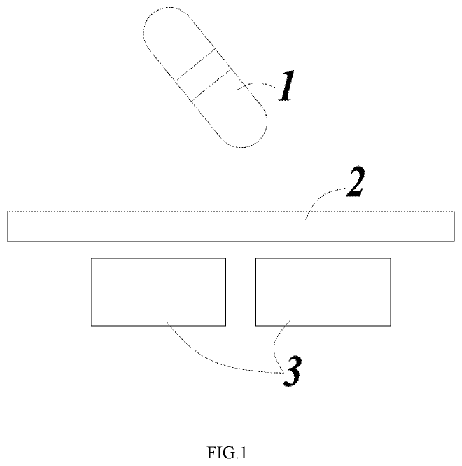

As shown in FIGS. 1 to 6, the present invention provides a positioning system and method for a swallowable device 1, used for positioning the in vivo swallowable device 1. The positioning system for the swallowable device 1 comprises a swallowable device 1 and a positioning device for detecting the position of the swallowable device 1 in the human digestive tract.

The positioning method comprises:

entering the m-th positioning period, wherein, m≥1;

energizing p electromagnetic coils simultaneously or sequentially, wherein, p≥2;

obtaining the actual magnetic field information Bwt sensed by the swallowable device each time the electromagnetic coils under energized state, wherein,1≤t≤p;

calculating the individual magnetic field information Bi at the swallowable device within the electromagnetic coils under separately energized state, wherein, 1≤i≤p;

detecting the pitch angle and roll angle of the swallowable device in the m-th positioning period;

calculating the yaw angle and spatial coordinates of the swallowable device in the m-th positioning period according to the pitch angle and roll angle and the individual magnetic field information Bi.

The swallowable device 1 comprises a magnetic field sensor for collecting magnetic field information, and the actual magnetic field information Bwt is detected and obtained by the magnetic field sensor. During the examination, a subject lies flat on the examination surface 2, at least two electromagnetic coils 3 are arranged, and the axes of the electromagnetic coils 3 are parallel to each other and perpendicular to the examination surface 2, so that the magnetic field lines of the at least two electromagnetic coils 3 are similar and more convenient to measure.

In the present invention, the position of the swallowable device 1 is determined by the magnetic field information sensed by the swallowable device 1, so that the individual magnetic field information Bi can be calculated according to the actual magnetic field information Bwt, and the individual magnetic field information Bi at the swallowable device 1 can be obtained when the electromagnetic coils 3 are separately energized, and then the attitude angle and spatial coordinates can be calculated. In addition, in this embodiment, it is necessary to detect the pitch angle and roll angle, but periodic calibration is impossible during detection of the yaw angle, so it is noisy. Therefore, the yaw angle and the spatial coordinates are calculated according to the individual magnetic field information Bi.

The pitch angle (pitch), roll angle (roll) and yaw angle (yaw) are all attitude angles. In the present invention, the positioning system for the swallowable device 1 adopts an attitude angle sensor to collect the pitch angle and roll angle. In an embodiment of the present invention, the attitude angle sensor uses an IMU chip.

In the present invention, the attitude angle sensor uses a 6-axis IMU chip. For the 6-axis IMU chip, only a 3-axis gyroscope and a 3-axis acceleration sensor are provided, so that the 6-axis IMU chip can only refer to the gravitational acceleration sensed by the acceleration sensor to periodically calibrate the pitch angle and roll angle, but cannot periodically calibrate the yaw angle. Therefore, the pitch angle and roll angle detected by the 6-axis IMU chip can be kept accurate, but the yaw angle cannot.

In addition, it should be noted that in the present invention, some letters are used to express some parameters, for example, individual magnetic field information Bi, actual magnetic field information Bwt, or spatial coordinates A as described below. And, the letters are bolded to indicate that these parameters are vectors. If an absolute value symbol is added to the above parameters, such as |Bi|, it means that the |Bi| is a scalar and a modulus of the individual magnetic field information Bi. If it refers to the component of an individual magnetic field information in a certain direction, such as B1_{X}, it is the component of the individual magnetic field information B1 on the x-axis.

According to the positioning method of the present invention, the individual magnetic field information Bi can be obtained by obtaining the actual magnetic field information Bwt sensed by the swallowable device 1 each time the electromagnetic coils under energized state. However, the geomagnetic field usually causes a certain error in the measurement and calculation of the individual magnetic field information Bi. If the positioning system for the swallowable device of the present invention uses a magnetic control device to move the swallowable device 1, the magnetic field of the magnetic control device can also have a greater impact on the measurement and calculation of the individual magnetic field information Bi. The magnetic field information sensed by the swallowable device 1 when none of the electromagnetic coils 3 are energized is called bottom magnetic field information Bg. The bottom magnetic field information Bg includes the geomagnetic field, and may also include the magnetic field generated by a magnetic control device. Then, the following two embodiments of the present invention can eliminate the influence of the bottom magnetic field information Bg in the calculation.

In the first embodiment of the present invention, before the step “calculating the individual magnetic field information Bi at the swallowable device within the electromagnetic coils under separately energized state” comprises:

In the m-th positioning period, detecting and obtaining the bottom magnetic field information Bg sensed by the swallowable device when the electromagnetic coils are not energized;

the step “calculating the individual magnetic field information Bi at the swallowable device within the electromagnetic coils under separately energized state” comprises:

calculating the individual magnetic field information Bi at the swallowable device within the electromagnetic coils under separately energized state according to the bottom magnetic field information Bg and the actual magnetic field information Bwt.

In the first embodiment, the bottom magnetic field information Bg is first measured and calculated, and in the subsequent calculation, the bottom field information Bg is subtracted from the actual magnetic field information Bwt. In addition, in the actual calculation method, it is related to the way the electromagnetic coils are energized, which is specified below by two embodiments.

In the first embodiment, the step “energizing p electromagnetic coils 3 simultaneously or sequentially” comprises:

conducting positive or negative pulse signals sequentially in the p electromagnetic coils 3, only at most one of the electromagnetic coils under energized state at the same time.

The step “calculating the individual magnetic field information Bi at the swallowable device within the electromagnetic coils 3 under separately energized state according to the bottom magnetic field information Bg and the actual magnetic field information Bwt” comprises:

Bi=Bwt−Bg, wherein, t=i.

Wherein, Bwt, namely Bwi, is the actual magnetic field information sensed by the swallowable device during the energizing of each electromagnetic coil 3. Positive or negative pulses are conducted in the p electromagnetic coils 3 in sequence. So, within the same positioning period, the magnitude and direction of the magnetic field sensed by the swallowable device 1 has p+1 states, one of which is that only the bottom magnetic field information Bg is sensed, and the others are the actual magnetic field information Bwi, wherein, 1≤i≤p. Also, in the actual magnetic field information Bwi, there is still included a part of the bottom magnetic field information Bg. Therefore, as long as the bottom magnetic field information Bg is subtracted from the actual magnetic field information Bwi, it is possible to obtain the individual magnetic field information Bi at the swallowable device 1 within the i-th electromagnetic coil 3 under energized state, thus facilitating subsequent calculations.

It should be noted that in the above step “conducting positive or negative pulse signals sequentially in the p electromagnetic coils 3”, the p electromagnetic coils 3 are not necessarily continuously conducting current pulses, but can also be conducted at intervals, and the p electromagnetic coils 3 do not conduct the current pulses between two stages of conducting the current pulses. This is also within the scope of the present invention.

Specifically, in the following description, take p=2 as an example, that is, the case where two electromagnetic coils 3 are sequentially energized is an embodiment. If p is any other number greater than 2, the object of the present invention can be achieved.

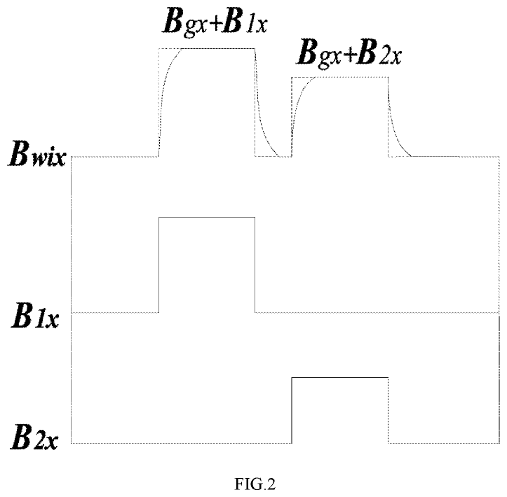

As shown in FIG. 2, there is a schematic diagram of the magnetic field changes of the actual magnetic field information Bwi and the individual magnetic field information B1 and B2 in the same direction, and the direction is assumed to be the x-axis. So, the number of times of energization of the two electromagnetic coils 3 which are energized sequentially is two.

And, specifically, the energization time of each electromagnetic coil 3 is Δt, and in this embodiment, as shown in FIG. 2, Δt is 150 ms. If it is assumed that at the beginning of the m-th stage, the time that the electromagnetic coil 3 is not energized is 120 ms. Therefore, the m-th positioning period consists of three stages: 1. Both of the two electromagnetic coils 3 are not energized and last for 120 ms; 2. The first electromagnetic coil 3 is energized and lasts for 150 ms; 3. The second electromagnetic coil 3 is energized and lasts for 150 ms. And, there is a 40 ms interval between the second stage and the third stage.

Then, it can be seen from FIG. 2 that the value of the actual magnetic field information Bwi_{X} in the first stage is equal to Bg_{X}, the value in the second stage is equal to Bg_{X}+B1_{X}, and the value in the third stage is equal to Bg_{X}+B2_{X}. Moreover, FIG. 2 only shows the magnitude change of the actual magnetic field information Bwi_{X} in one direction. Of course, in the three-axis direction, the magnitude change and calculation method of the actual magnetic field information Bwi_{X} are also similar to the above, and cannot be repeated here.

Further, since current pulses are applied to the electromagnetic coils 3, and the electromagnetic coils 3 themselves have a transient response, it is necessary to wait for a certain period of time before the actual magnetic field information Bwi becomes stable. Therefore, the time of applying current pulses to the electromagnetic coils 3 needs to be longer than the time of the transient response. The time length of the transient response is determined by the coil inductance L and the resistance R. The time constant of the first order circuit step response is

$$τ = \frac{L}{R} .$$

The transient response time is usually 5τ, so it is about 5˜30 ms. Therefore, in this embodiment, the m-th positioning period lasts for a total of 500 ms. Also, the m-th positioning period can be of other lengths, as long as it is sufficient for the swallowable device 1 to sense the actual magnetic field information Bwi, it can fall within the scope of the present invention.

In the second embodiment of the present invention, the step “energizing p electromagnetic coils 3 simultaneously or sequentially” comprises:

conducting periodic signals in the p electromagnetic coils simultaneously, and the current frequency conducted in the electromagnetic coils is different.

The step “calculating the individual magnetic field information Bi at the swallowable device within the electromagnetic coils under separately energized state” includes: calculating the magnetic field information Bwt by Fourier decomposition to obtain the individual magnetic field information Bi at the swallowable device within the p electromagnetic coils under separately energized state.

In the second embodiment, the p electromagnetic coils 3 are energized simultaneously, and all conduct periodic signals , so that Fourier decomposition can be performed. For this m-th positioning period, the electromagnetic coils 3 are energized only once, so the actual magnetic field information sensed by the swallowable device 1 every time the electromagnetic coils 3 are energized is Bw1. Fourier decomposition is performed on the magnetic field information Bw1 to obtain individual magnetic field information Bi, and the bottom magnetic field information Bg can also be decomposed. Specifically, Bw1 in a certain time window can be selected in the m-th positioning period, and the width of the time window is short enough so that the bottom magnetic field information Bg in this time window does not change substantially. Thus, he bottom magnetic field information Bg can be eliminated from the actual magnetic field information Bw1 by Fourier decomposition.

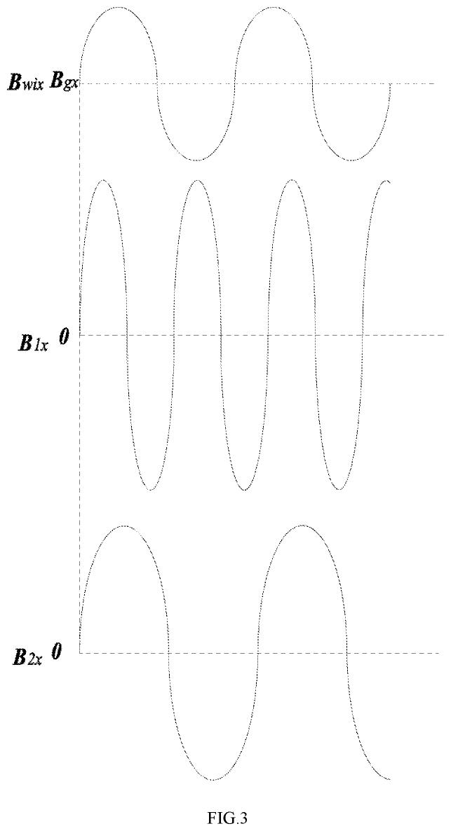

As shown in FIG. 3, similarly, schematically showing the change of the following magnetic field in one direction. Specifically, it is assumed that the direction is the x-axis direction. Specifically, it is, from top to bottom, the actual magnetic field information Bw1_{X}, sensed by the swallowable device 1 within the electromagnetic coil 3 under energized state, the magnetic field information B1_{X} at the swallowable device 1 within the first electromagnetic coil 3 under energized state, and the magnetic field information B2_{X} at the swallowable device 1 within the second electromagnetic coil 3 under energized state. Wherein, Bw1_{X}=Bg_{X}+B1_{X}+B2_{X}.

Since the frequency of the current passing through the first electromagnetic coil 3 and the second electromagnetic coil 3 is different, the actual magnetic field information Bw1_{X} can be analyzed by Fourier decomposition, to obtain the magnetic field information B1_{X} and B2_{X}. In this embodiment, it is only needed to detect the actual magnetic field information Bw1 once, then the magnetic field information B1 and B2 of the two electromagnetic coils 3 can be measured at the same time, which is more convenient and can shorten the positioning period accordingly. In the above steps, only the magnetic field change in one direction is shown and calculated. If in the three-axis directions, the magnitude changes and the calculation method of the actual magnetic field information Bwi_{X} are also similar to the above, they cannot be repeated here.

The above describes the positioning method when the p electromagnetic coils are energized simultaneously, or conduct positive or negative currents sequentially. The following can describe another embodiment that can avoid the influence of the bottom magnetic field information Bg.

In the second embodiment, it is unnecessary to measure the bottom magnetic field information Bg. Instead, offset the positive and negative signals during conducting square wave pulse signals in the electromagnetic coils, so as to eliminate the influence of the bottom magnetic field information Bg in the calculation.

Specifically, in the third embodiment, the step “energizing p electromagnetic coils simultaneously or sequentially” comprises:

controlling to conduct square-wave pulse signals in the p electromagnetic coils sequentially, the absolute values of high level and low level in the same square-wave pulse signal are equal, and only at most one of the electromagnetic coils under energized state at the same time.

The step “obtaining the actual magnetic field information Bwt sensed by the swallowable device each time the electromagnetic coils under energized state” comprises:

obtaining the actual magnetic field information Bwt₊, sensed by the swallowable device each time the electromagnetic coils conducts a positive current;

obtaining the actual magnetic field information Bwt₋, sensed by the swallowable device each time the electromagnetic coils conducts a negative current.

The step “calculating the individual magnetic field information Bi at the swallowable device within the electromagnetic coils under separately energized state” comprises:

$$B i = \frac{B w {t}_{+} - {Bwt}_{-}}{2} ,$$

wherein, t=i.

In the third embodiment described above, conduct square-wave pulse signals in the electromagnetic coils 3 sequentially, and the absolute values of high level and low level are equal, the actual magnetic field information Bwt₊ and Bwt₋ both include the bottom magnetic field information Bg, so that the individual magnetic field information Bi of the electromagnetic coils 3 under energized can be obtained by subtracting the positive and negative actual magnetic field information Bwt from each other in subsequent calculation.

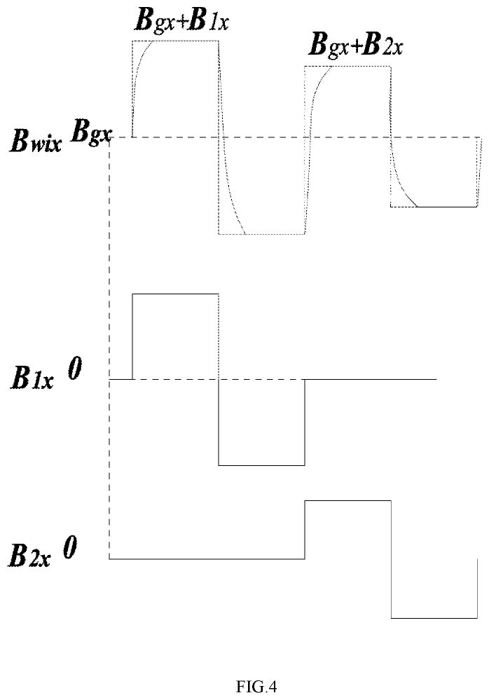

Specifically, assuming that p is 2, that is, the number of electromagnetic coils 3 is two. As shown in FIG. 4, showing the changes in values of the individual magnetic field information B1, B2 and the actual magnetic field information Bwi of the two electromagnetic coils 3 in the x direction. The individual magnetic field information when the two electromagnetic coils conduct positive current is B1_{X} and B2_{X}, and |B2_{X}| is less than |B1_{X}|. Then, the actual magnetic field information Bwt sensed by the swallowable device 1 within the electromagnetic coils 3 under energized state is specifically:

Bw1_{X+}=Bg_{X}+B1_{X}, and B1_{X} is positive;

Bw1_{X-}=Bg_{X}+B1_{X}, and B1_{X} is negative;

Bw2_{X+}=Bg_{X}+B2_{X}, and B2_{X} is positive;

Bw2_{X-}=Bg_{X}+B2_{X}, and B2_{X} is negative.

It can be seen that in the x-axis direction, B1_{X}=(Bw1_{X+}−Bw1_{X-})/2, B2_{X}=(Bw2_{X+}−Bw2_{X-})/2. Since both B1 and B2 are square waves, the change in magnitude of B1 and B2 in the x-axis direction can be obtained by the above calculation.

In the above steps, only the magnetic field change in one direction is shown and calculated. If in the three-axis directions, the magnitude changes and the calculation method of the actual magnetic field information Bwi_{X} are also similar to the above, they cannot be repeated here. Therefore, by subtracting the actual magnetic field information Bwt₊ from Bwt₋ when the same electromagnetic coil is energized, the bottom magnetic field information Bg can be eliminated from the actual magnetic field information Bwt, and information on the amplitude change of B1 and B2 along time can be obtained. In this embodiment, there is no need to measure the bottom magnetic field information Bg, which further simplifies the steps.

In the fourth embodiment, the step “obtaining the actual magnetic field information Bwt sensed by the swallowable device each time the electromagnetic coils under energized state” comprises:

applying square-wave pulse signals to the p electromagnetic coils simultaneously, the absolute values of the high level and low level in the same square-wave pulse signal are equal, and the phases of square waves applied to the electromagnetic coils are different.

The step “calculating the individual magnetic field information Bi at the swallowable device within the electromagnetic coils under separately energized state” comprises:

decomposing the actual magnetic field information Bwt according to the difference in amplitude, and obtaining Bwtₙ in turn; wherein, 1≤n≤2p;

$$B i = \frac{{Bwt}_{j} - Bw {t}_{k}}{2} ,$$

wherein, j and k belong to n.

In this fourth embodiment, to obtain different individual magnetic field information Bi, the signals of different amplitudes in the obtained actual magnetic field information Bwt can also be subtracted and divided by two.

Since, in this embodiment, square wave pulse signals with different phases are applied simultaneously to the p electromagnetic coils, the amplitude of the actual magnetic field information Bwt changes significantly after the square wave signals are superimposed, and the amplitude values of the p electromagnetic coils are all different, so that 2p amplitude values can be obtained.

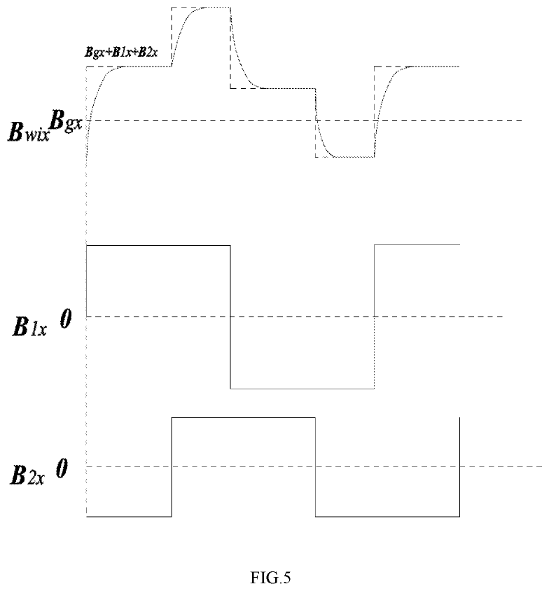

Specifically, as shown in FIG. 5, from top to bottom is the change in magnitude of the actual magnetic field information Bwt, the individual magnetic field information B1 of the coil 1, and the individual magnetic field information B2 of the coil 2 in the x-axis direction, respectively.

Since, the phases of the individual magnetic field information B1 and B2 are different, the amplitude of the actual magnetic field information Bwt varies greatly. Specifically, as shown in FIG. 5, a cycle of the actual magnetic field information BwtX is divided into four stages, from BwtX₁ to BwtX₄.

Wherein, Bwt1_{X}=Bg_{X}+B1_{X}+B2_{X}, in the stage i=1, B1_{X} is positive and B2_{X} is negative; in the stage i=2, B1_{X} is positive and B2_{X} is positive; in the stage i=3, B1_{X} is negative and B2_{X} is positive; in the stage i=4, B1_{X} is negative and B2_{X} is negative.

Thus,

$$B {1}_{X} = \frac{B w t {1}_{X} - B w t {2}_{X}}{2} ; B {2}_{X} = \frac{B M {2}_{X} - B w t {1}_{X}}{2} .$$

Therefore, the bottom magnetic field information Bg_{X} can be eliminated from the actual magnetic field information Bwt_{X}, and information on the amplitude change of B1_{X} and B2_{X} along time can be obtained. Similarly, in the above steps, only the magnetic field change in one direction is shown and calculated. If in the three-axis directions, the magnitude changes and the calculation method of the actual magnetic field information Bwt_{X} are also similar to the above, they cannot be repeated here.

If there are three or more electromagnetic coils, the actual magnetic field information Bwt can be divided into 2p stages according to the amplitude changes in stages. In addition, the values of j and k can be selected according to the waveforms of the actual magnetic field information Bwt, and cannot be repeated here.

Further, the spatial coordinates and yaw angle Yaw can be calculated once the individual magnetic field information Bi is known. Specifically, the step “calculating the yaw angle and spatial coordinates of the swallowable device in the m-th positioning period according to the pitch angle and roll angle and the individual magnetic field information Bi” comprises:

establishing a magnetic field model for the electromagnetic coil 3, and the magnetic field model is:

$${B}_{t} = \int \frac{{μ}_{0} I}{4 π} \frac{dI \times r}{{r}^{3}} . \qquad ( Formula 1 )$$

Wherein, μ₀ is the vacuum permeability, I is the current intensity in the electromagnetic coil 3 and corresponds to the measured individual magnetic field information Bi, dl is the current element, r is the vector from the current element to the swallowable device 1, r is the distance from the current element to the swallowable device.

Calculating the estimated magnetic field information Bti at the location of the swallowable device within the electromagnetic coils 3 under energized state according to Formula 1.

Establishing a spatial coordinates calculation model, that is:

$${argmin}_{x , y , z , Yaw} {\sum }_{i = 1}^{p} {‖ {R}_{r} {R}_{p} R ( Yaw ) {B}_{i} - {B}_{ti} ( x , y , z ) ‖}_{2}^{2} , \qquad ( Formula 2 )$$

Wherein, Rᵣ is the rotation matrix corresponding to the roll angle, Rₚ is the rotation matrix corresponding to the pitch angle, R_{Yaw} is the rotation matrix corresponding to the yaw angle; and the symbol ∥ ∥₂² denotes the square of the two norm of the vector, that is, the sum of the squares of the elements of the vector and p is the number of coils, so p≥2.

Performing an optimized solution algorithm on the calculation model in Formula 2, calculating the yaw angle and spatial coordinates of the swallowable device in the m-th positioning period.

Wherein, the magnetic field model in Formula 1 is modeled by Biot Savart's law.

Through the above process, in the case where the yaw angle Yaw cannot be accurately measured, the yaw angle Yaw and the spatial coordinates of the swallowable device 1 in the m-th period can also be obtained.

Since, when the swallowable device 1 moves in the human intestinal tract, if it moves over a large area in that m-th positioning period, the position of the swallowable device 1 cannot be measured accurately. Therefore, the duration of the positioning period should not be too long to prevent inaccurate measurement. Moreover, the positioning method disclosed herein also includes a determination of whether the swallowable device is moving.

Specifically, before the step “detecting the pitch angle and roll angle of the swallowable device in the m-th positioning period” comprises:

determining whether the swallowable device 1 moves in the m-th positioning period;

if the swallowable device does not move in the m-th positioning period, enter the detection or calculation of the pitch angle and roll angle;

if the swallowable device moves in the m-th positioning period, abandon the m-th positioning period and enter the m+1-th positioning period.

That is, before the detection and calculation of the pitch angle and the roll angle, it is determined whether the swallowable device 1 moves during this m-th positioning period. If it moves, the individual magnetic field information Bi is not accurate enough to be used as a reference for the final spatial coordinates, so this m-th positioning period is abandoned while entering the m+1-th positioning period to restart detection and calculation. If it does not move, the individual magnetic field information Bi is accurate, and subsequent detection or calculation can be performed.

In this embodiment, the step “determining whether the swallowable device 1 moves in the m-th positioning period” comprises:

counting the vibration amplitude of the acceleration value of the swallowable device 1 in the m-th positioning period;

if the vibration amplitude is greater than the threshold S, it is determined that the capsule moves;

if the vibration amplitude is less than or equal to the threshold S, it is determined that the capsule does not move.

That is, in this embodiment of the present invention, the vibration amplitude of the acceleration value of the swallowable device 1 during the m-th positioning period is used to determine whether the capsule moves or not. The vibration amplitude of the acceleration value in the m-th positioning period is the difference between the maximum and minimum acceleration values. If the vibration amplitude is too great, it is clear that the swallowable device 1 moves, so skip the m-th positioning period and enter the m+1-th positioning period for detection and calculation. Other methods, such as installing other units for measuring speed, angular velocity or other parameters in the swallowable device 1 to determine whether the swallowable device 1 moves, are also within the protection scope of the present invention.

Moreover, since the positioning period is usually short, the position of the swallowable device 1 in consecutive positioning periods usually has consecutiveness. Therefore, the spatial coordinates of the swallowable device 1 can be predicted.

Specifically, the positioning method in the present invention further comprises:

obtaining the spatial coordinates of the swallowable device 1 in the m-th, m+1-th, and m+2-th positioning periods, and recording them as A, B, and C respectively;

predicting the spatial coordinates D′ of the swallowable device 1 in the m+3-th positioning period;

entering the m+3-th positioning period, and calculating the spatial coordinates D of the swallowable device 1 in the m+3-th positioning period;

calculating the distance d between D and D′ in three-dimensional space;

if d<threshold L, using D as the spatial coordinates of the swallowable device 1 in the m+3-th positioning period;

if d>threshold L, using D′ as the spatial coordinates of the swallowable device 1 in the m+3-th positioning period.

That is, by determining the position of the swallowable device 1 in the first three consecutive positioning periods, the position of the swallowable device 1 in the m+3-th positioning period can be predicted. If the actual spatial coordinates in the m+3-th positioning period are too far away from the predicted spatial coordinates D′, it means that the spatial coordinates in the m+3-th positioning period are incorrectly calculated, so the predicted spatial coordinates D′ are taken as the spatial coordinates in the m+3-th positioning period. In this embodiment, the threshold L is 2 mm, that is, the spatial coordinates D obtained by calculation must be within a 2 mm spherical space around the predicted spatial coordinates D′ before it can be determined as the spatial coordinates of the swallowable device 1 in the m+3-th positioning period.

The step “predicting the spatial coordinates D′ of the swallowable device 1 in the m+3-th positioning period” comprises:

D′=C+2BC−AB , wherein, BC and AB are both vectors.

Since, in the consecutive positioning periods, the position of the swallowable device 1 can be determined as follows:

CD−BC≈BC−AB;

Then the position of D is:

D=C+CD,

D=C+2BC−AB.

Therefore, the position of the swallowable device 1 in the m+3-th positioning period can be predicted by the above method, and the calculated spatial coordinates D of the swallowable device 1 can be filtered by the predicted spatial coordinates D′, to eliminate the points that disrupt the movement trajectory of the swallowable device 1, so that the movement trajectory presents a smooth curve, which is also consistent with the actual situation of the movement of the swallowable device 1 in the digestive tract.

If, as described above, there are no spatial coordinates in a certain positioning period because the swallowable device 1 moves in the positioning period, the spatial coordinates in the positioning period can be estimated according to the spatial coordinates in the positioning periods before and after said positioning period to get an estimated value. Likewise, this can also make the trajectory of the swallowable device 1 present a continuous smooth curve.

In addition, since the magnetic field strength of the electromagnetic coil 3 decreases with increasing distance, when the swallowable device 1 is too far away from a certain electromagnetic coil 3, the magnetic field signal Bwt sensed by the swallowable device 1 decreases significantly, which can result in a decrease in the signal-to-noise ratio and a decrease in the positioning accuracy. Therefore, after measuring the intensity of the individual magnetic field Bi in the previous m-th positioning period, the magnitude of the current pulses through the electromagnetic coils 3 in the m+1-th positioning period can be adjusted.

Specifically, the positioning method further comprises:

determining the magnitude of the individual magnetic field information Bi in the m-th period;

if gₕ<|Bi|, reducing the current of the electromagnetic coil 3i in the m+1-th period;

if gₗ≤|Bi|≤gₕ, keeping the current of the electromagnetic coil 3i in the m+th period;

if |Bi|<gₗ, increasing the current of the electromagnetic coil 3i in the m+1-th period.

Wherein, gl is 100 uT and gh is 500 uT. Within this range, the current conducted in the electromagnetic coil 3 is adaptively adjusted, and the magnitude of the magnetic field of the electromagnetic coil 3 is adjusted accordingly, so as to ensure the accuracy of positioning. In addition, the magnetic field strength of the electromagnetic coil 3 should not be too strong. For one thing, according to the aspects of the present invention, the swallowable device 1 can be provided with a magnet to be controlled to move by the magnetic control device. If the magnetic field intensity of the electromagnetic coil 3 is too strong, it may cause the swallowable device 1 to shake, and thus result in problems with the clarity of the captured images and the positioning accuracy. For another, if the current in the electromagnetic coil 3 is too high, the power consumption can also increase, and the heating of the electromagnetic coil 3 can increase.

In addition, in the step “energizing p electromagnetic coils 3 simultaneously or sequentially”, the quantity of the electromagnetic coils 3 can not only be set to p, but it can also set to n. Among the n electromagnetic coils 3, select p electromagnetic coils 3 to energize simultaneously or sequentially, wherein, n>p. Therefore, the positioning method further comprises: changing the position or quantity of the energized electromagnetic coils 3 in different positioning periods according to the position of the swallowable device 1. Since, as described above, if the distance between the swallowable device 1 and the electromagnetic coil 3 is too far, the magnetic field intensity sensed by the swallowable device 1 can be too weak, which can affect the measurement of spatial coordinates of the swallowable device 1. Thus, in this embodiment, n electromagnetic coils 3 may be provided, and in different positioning periods, different electromagnetic coils 3 are selected according to the position of the swallowable device 1 to perform energization operation as above.

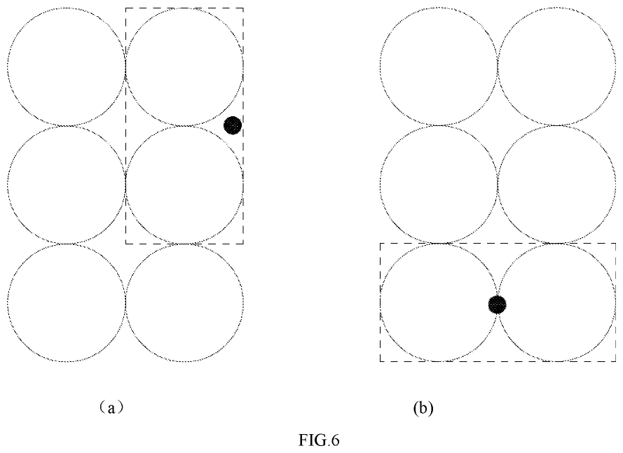

Specifically, as shown in FIG. 6, from left to right, showing the position distribution of the swallowable device 1 in two different positioning periods, and the black dots in the figure refer to the projection of the swallowable device 1 on the plane where the coils are located. It can be seen from the figure that if the swallowable device 1 is located in the position of FIG. 6(a), the electromagnetic coils 3 to be energized are those in the dashed box in FIG. 6(a); if the swallowable device 1 is located in the position of FIG. 6(b), the electromagnetic coils 3 to be energized are those in the dashed box in FIG. 6(b).

Also, the electromagnetic coils 3 can be arranged either in a straight line or in a matrix. If the electromagnetic coils 3 are arranged in a straight line and there are n coils in total, adjacent electromagnetic coils 3 can be selected as the ones to be activated in the m-th positioning period according to the position of the swallowable device 1; if the electromagnetic coils 3 are arranged in a matrix and there are n coils in total, the electromagnetic coils 3 arranged horizontally, longitudinally or diagonally can be selected as the ones to be activated in the m-th positioning period according to the position of the swallowable device 1.

Since the swallowable device 1 moves slowly in the digestive tract, the electromagnetic coils 3 activated in the adjacent positioning periods are not too far apart. Therefore, the position or quantity of the swallowable device 1 activated in this positioning period can be changed according to the position or quantity of the swallowable device 1 activated in the previous positioning period, so as to adaptively switch the electromagnetic coils 3. In this embodiment, the distance between the energized electromagnetic coil 3 and the swallowable device 1 is relatively close, so the current conducted in the electromagnetic coil 3 does not need to be too high, which reduces power consumption and heat generation of the electromagnetic coil 3.

The present invention further provides a positioning system for the swallowable device 1 using the method described above. The system comprises a swallowable device 1 and a positioning device for detecting the position of the swallowable device 1. The swallowable device 1 comprises an enclosure, a sensor and a magnetic field sensor arranged in the enclosure for collecting magnetic field information. The positioning device comprises an examination surface 2 and at least two electromagnetic coils 3. The axes of the electromagnetic coils 3 are parallel to each other and arranged perpendicular to the examination surface 2. The sensor is an attitude angle sensor for detecting the attitude angle of the swallowable device 1, or an acceleration sensor for detecting the motion state of the swallowable device 1. The specific functions and roles are as described above, and cannot be repeated here.

Specifically, the swallowable device further comprises a magnet, and the positioning device further comprises a magnetic control device for controlling the magnet. Thus, the magnetic control device can control the magnet and drive the swallowable device 1 to move. Details are not repeated here.

In addition, the electromagnetic coils 3 are located on the side of the examination surface 2 away from the swallowable device 1, and the magnetic control device and the swallowable device 1 are on the same side of the examination surface 2.

The electromagnetic coils 3 can be arranged in a straight line, so the quantity of electromagnetic coils 3 is at least two, and they are arranged along the height direction of the human body. Alternatively, the electromagnetic coils 3 can also be arranged in a matrix, so the quantity of the electromagnetic coils 3 is at least four. In this embodiment, the electromagnetic coils 3 to be energized in different positioning periods can be arranged longitudinally or horizontally.

Further, the positioning system for the swallowable device also comprises a cooling device that is arranged between the examination surface 2 and the electromagnetic coils 3. Since the electromagnetic coils 3 generate heat after being energized, while the heat can increase the internal resistance of the electromagnetic coils 3 and also affect the user, a cooling device can be provided to prevent the electromagnetic coils 3 from overheating.

In summary, the present invention relates to a positioning system and method for a swallowable device 1 in the human digestive tract. In this positioning method, the actual magnetic field information Bwt is measured first, and the influence of the bottom magnetic field information Bg on the spatial coordinate measurement of the swallowable device 1 can be eliminated in the subsequent measurement. In addition, two specific embodiments and four different examples are proposed to calculate the individual magnetic field information Bi, so that the calculation of the individual magnetic field information Bi is more accurate, and different methods are also used to improve the efficiency of the calculation.

It should be understood that, although the specification is described in terms of embodiments, not every embodiment merely includes an independent technical solution. This narration in the specification is only for clarity. Those skilled in the art should have the specification as a whole, and the technical solutions in each embodiment may also be combined as appropriate to form other embodiments that can be understood by those skilled in the art.

The series of detailed descriptions listed above are only specific descriptions of the feasible embodiments of the present invention, and are not intended to limit the protection scope of the present invention. Any equivalent embodiments or variations made without departing from the technical spirit of the present invention should be included in the protection scope of the present invention.

1. A positioning method for a swallowable device, used for positioning the in vivo swallowable device, comprising:
entering the m-th positioning period, wherein, m≥1;
energizing p electromagnetic coils simultaneously or sequentially, wherein, p≥2;
obtaining the actual magnetic field information Bwt sensed by the swallowable device each time the electromagnetic coils under energized state, wherein,1≤t≤p;
calculating the individual magnetic field information Bi at the swallowable device within the electromagnetic coils under separately energized state, wherein, 1≤i≤p;
detecting the pitch angle and roll angle of the swallowable device in the m-th positioning period;
calculating the yaw angle and spatial coordinates of the swallowable device in the m-th positioning period according to the pitch angle and roll angle and the individual magnetic field information Bi;
wherein before the step “detecting the pitch angle and roll angle of the swallowable device in the m-th positioning period” comprises:
determining whether the swallowable device moves in the m-th positioning period;
entering the detection or calculation of the pitch angle and roll angle when the swallowable device does not move in the m-th positioning period;
entering the m+1-th positioning period when the swallowable device moves in the m-th positioning period.

2. The positioning method of claim 1, wherein before the step “calculating the individual magnetic field information Bi at the swallowable device within the electromagnetic coils under separately energized state” comprises:
detecting and obtaining the bottom magnetic field information Bg sensed by the swallowable device when the electromagnetic coils are not energized in the m-th positioning period; and
the step “calculating the individual magnetic field information Bi at the swallowable device within the electromagnetic coils under separately energized state” comprises:
calculating the individual magnetic field information Bi at the swallowable device within the electromagnetic coils under separately energized state according to the bottom magnetic field information Bg and the actual magnetic field information Bwt.

3. The positioning method of claim 2, wherein the step “energizing p electromagnetic coils simultaneously or sequentially” comprises:
conducting positive or negative pulse signals sequentially in the p electromagnetic coils, at most one of the electromagnetic coils under energized state at the same time; and
the step “calculating the individual magnetic field information Bi at the swallowable device within the electromagnetic coils under separately energized state according to the bottom magnetic field information Bg and the actual magnetic field information Bwt” comprises:
Bi=Bwt−Bg, wherein, t=i.

4. The positioning method of claim 1, wherein the step “energizing p electromagnetic coils simultaneously or sequentially” comprises:
conducting periodic signals in the p electromagnetic coils simultaneously, and the current frequency conducted in the electromagnetic coils is different; and
the step “calculating the individual magnetic field information Bi at the swallowable device within the electromagnetic coils under separately energized state” comprises:
calculating the magnetic field information Bwt by Fourier decomposition to obtain the individual magnetic field information Bi at the swallowable device within the p electromagnetic coils under separately energized state.

5. The positioning method of claim 1, wherein the step “energizing p electromagnetic coils simultaneously or sequentially” comprises:
controlling to conduct square-wave pulse signals in the p electromagnetic coils sequentially, the absolute values of high level and low level in the same square-wave pulse signal are equal, and at most one of the electromagnetic coils under energized state at the same time; and
the step “obtaining the actual magnetic field information Bwt sensed by the swallowable device each time the electromagnetic coils under energized state” comprises:
obtaining the actual magnetic field information Bwt₊, sensed by the swallowable device each time the electromagnetic coils conducts a positive current;
obtaining the actual magnetic field information Bwt₋, sensed by the swallowable device each time the electromagnetic coils conducts a negative current; and
the step “calculating the individual magnetic field information Bi at the swallowable device within the electromagnetic coils under separately energized state” comprises:
$$B i = \frac{B w {t}_{+} - {Bwt}_{-}}{2} ,$$
wherein, t=i.

6. The positioning method of claim 1, wherein the step “obtaining the actual magnetic field information Bwt sensed by the swallowable device each time the electromagnetic coils under energized state” comprises:
applying square-wave pulse signals at same frequencies to the p electromagnetic coils simultaneously, the absolute values of the high level and low level in the same square-wave pulse signal are equal, and the phases of square waves applied to the electromagnetic coils are different; and
the step “calculating the individual magnetic field information Bi at the swallowable device within the electromagnetic coils under separately energized state” comprises:
decomposing the actual magnetic field information Bwt according to the difference in amplitude, and obtaining Bwtₙ in turn; wherein, 1≤n≤2p;
$$B i = \frac{{Bwt}_{j} - Bw {t}_{k}}{2} ,$$
wherein, j and k belong to n.

7. The positioning method of claim 1, wherein the step “calculating the yaw angle and spatial coordinates of the swallowable device in the m-th positioning period according to the pitch angle and roll angle and the individual magnetic field information Bi” comprises:
establishing a magnetic field model for the electromagnetic coil, and the magnetic field model is:
$${B}_{t} = \int \frac{{μ}_{0} I}{4 π} \frac{dI \times r}{{r}^{3}} , \qquad ( Formula 1 )$$
wherein, μ₀ is the vacuum permeability, I is the current intensity in the electromagnetic coil and corresponds to the measured individual magnetic field information Bi, dl is the current element, r is the vector from the current element to the swallowable device, r is the distance from the current element to the swallowable device;
calculating the estimated magnetic field information Bti at the location of the swallowable device within the electromagnetic coils under energized state according to Formula 1;
establishing a spatial coordinates calculation model, that is:
$${argmin}_{x , y , z , Yaw} {\sum }_{i = 1}^{p} {‖ {R}_{r} {R}_{p} R ( Yaw ) {B}_{i} - {B}_{ti} ( x , y , z ) ‖}_{2}^{2} , \qquad ( Formula 2 )$$
wherein, Rᵣ is the rotation matrix corresponding to the roll angle, Rₚ is the rotation matrix corresponding to the pitch angle, R_{Yaw} is the rotation matrix corresponding to the yaw angle;
performing an optimized solution algorithm on the calculation model in Formula 2, and calculating the yaw angle and spatial coordinates of the swallowable device in the m-th positioning period.

8. (canceled)

9. The positioning method of claim 1, wherein the step “determining whether the swallowable device moves in the m-th positioning period” comprises:
counting the vibration amplitude of the acceleration value of the swallowable device in the m-th positioning period;
determining that the swallowable device moves when the vibration amplitude is greater than the threshold S;
determining that the swallowable device does not move when the vibration amplitude is less than or equal to the threshold S.

10. The positioning method of claim 1, wherein the method further comprises:
obtaining the spatial coordinates of the swallowable device in the m-th, m+1-th, and m+2-th positioning periods, and recording them as A, B, and C respectively;
predicting the spatial coordinates D′ of the swallowable device in the m+3-th positioning period;
entering the m+3-th positioning period, and calculating the spatial coordinates D of the swallowable device in the m+3-th positioning period;
calculating the distance d between D and D′ in three-dimensional space;
if d<threshold L, using D as the spatial coordinates of the swallowable device in the m+3-th positioning period;
if d>threshold L, using D′ as the spatial coordinates of the swallowable device in the m+3-th positioning period.

11. The positioning method of claim 10, wherein the step “predicting the spatial coordinates D′ of the swallowable device in the m+3-th positioning period” comprises:
D′=C+2BC−AB, wherein, BC and AB are both vectors.

12. The positioning method of claim 1, wherein the method further comprises:
determining the magnitude of the individual magnetic field information Bi in the m-th period;
if gₕ<|Bi|, reducing the current of the electromagnetic coil i in the m+1-th period;
if gₗ<|Bi|<gₕ, keeping the current of the electromagnetic coil i in the m+1-th period;
if |Bi|<gₗ, increasing the current of the electromagnetic coil i in the m+1-th period.

13. The positioning method of claim 1, wherein the step “energizing p electromagnetic coils simultaneously or sequentially” comprises:
selecting p electromagnetic coils from n electromagnetic coils and energizing simultaneously or sequentially, wherein, n>p;
the positioning method further comprises:
changing the position or quantity of the energized electromagnetic coils in different positioning periods according to the position of the swallowable device.

14. A positioning system for a swallowable device using the method of claim 1, used for positioning the in vivo swallowable device, comprising a swallowable device and a positioning device for detecting the position of the swallowable device, wherein the swallowable device comprises an enclosure, a sensor arranged in the enclosure and a magnetic field sensor for collecting magnetic field information; the positioning device comprises an examination surface and at least two electromagnetic coils; the sensor is an attitude angle sensor for detecting the attitude angle of the swallowable device, or an acceleration sensor for detecting the motion state of the swallowable device.

15. The positioning system of claim 14, wherein the swallowable device further comprises a magnet, and the positioning device further comprises a magnetic control device for controlling the magnet.

16. The positioning system of claim 15, wherein the electromagnetic coils are located on the side of the examination surface away from the swallowable device, and the magnetic control device and the swallowable device are on the same side of the examination surface.

17. The positioning system of claim 14, wherein the electromagnetic coils are arranged in a straight line.

18. The positioning system of claim 14, wherein the electromagnetic coils are arranged in a matrix.

19. The positioning system of claim 14, wherein the system comprises a cooling device that is arranged between the examination surface and the electromagnetic coils.
